(19) United States
(12) Patent Application Publication
(10) Pub. No.: US 2019/0276896 A1
(43) Pub. Date: Sep. 12, 2019

(54) METHODS AND MATERIALS FOR DETECTING GENE AMPLIFICATION

(71) Applicant: Mayo Foundation for Medical Education and Research, Rochester, MN (US)

(72) Inventors: Andre M. Oliveira, Rochester, MN (US); Michele R. Erickson-Johnson, Rochester, MN (US)

(73) Assignee: Mayo Foundation for Medical Education and Research, Rochester, MN (US)

(21) Appl. No.: 16/272,893

(22) Filed: Feb. 11, 2019

Related U.S. Application Data
(63) Continuation of application No. 15/380,883, filed on Dec. 15, 2016.
Application No. 15/380,883 is a continuation of application No. 14/549,216, filed on Nov. 20, 2014, now abandoned.
Application No. 14/549,216 is a continuation of application No. 12/763,756, filed on Apr. 20, 2010, now abandoned.
(60) Provisional application No. 61/214,343, filed on Apr. 22, 2009.

(51) Int. Cl. C12Q 1/6886; C12Q 1/6883; C12Q 1/6841

(52) U.S. Cl. CPC C12Q 1/6886; C12Q 1/6883; C12Q 2600/112; C12Q 2600/106; C12Q 2600/158; C12Q 1/6841

(57) ABSTRACT

This document relates to methods and materials involved in detecting gene amplification in a mammal. For example, methods and materials for detecting amplification at CPM and MDM2 loci to determine the presence or absence of a malignant lipomatous neoplasm in a mammal are provided.

### CROSS-REFERENCE TO RELATED APPLICATIONS

This application is a continuation of U.S. Ser. No. 15/380,883, filed Dec. 15, 2016, which is a continuation of U.S. Ser. No. 14/549,216, filed Nov. 20, 2014 (Abandoned), which is a continuation of U.S. Ser. No. 12/763,756, filed Apr. 20, 2010 (Abandoned), which claims priority to U.S. Provisional Application No. 61/214,343 filed on Apr. 22, 2009, the contents of which are incorporated by reference herein in their entirety.

### BACKGROUND

### 1. Technical Field

This document relates to methods and materials involved in detecting gene amplification in mammals. For example, this document provides methods and materials for detecting amplification at CPM and MDM2 loci to determine the presence or absence of a malignant lipomatous neoplasm in mammals.

### 2. Background Information

Soft tissue tumors are a large and heterogeneous group of neoplasms. The broad spectrum of soft tissue tumors exhibiting adipose tissue differentiation includes ordinary lipomas and subtypes, liposarcomas and subtypes, and hibernomas. Classification of soft tissue tumors is made mainly according to histologic and immunophenotypic parameters. Cytogenetic and molecular genetic analysis have been more frequently used to better classify these tumors. It can be difficult to distinguish benign and malignant soft tissue neoplasms using traditional histological, and this issue could not be better exemplified with lipomatous neoplasms. For example, histologic exam can be inadequate to distinguish ordinary lipomas, which are benign mesenchymal neoplasms, from well-differentiated liposarcoma/atypical lipomatous tumors (WDL/ALT), which are locally aggressive malignant mesenchymal neoplasms. Consequently, reliance only upon such traditional methods may lead to erroneous diagnosis and inadequate treatments.

### SUMMARY

This document provides methods and materials involved in detecting gene amplification and distinguishing benign from malignant lipomatous neoplasms in a sample from a mammal (e.g., a human) on the basis of such gene amplification. For example, this document provides nucleic acids for detecting gene amplifications present on ring and/or giant rod chromosomes. Such nucleic acids can be used to detect CPM gene amplification, MDM2 gene amplification, or both. In some cases, such nucleic acids can be used to identify CDK4, and TSPAN31 gene amplifications. As described herein, the methods and materials provided can be used for tumor cytogenetic diagnosis and detection of aberrant gene amplification. Evaluating amplification of aberrant gene expression according to the methods provided herein can allow a pathologist or a molecular pathologist to better discriminate normal adipose tissue/lipoma from atypical lipomatous tumor/well-differentiated liposarcoma. Such analytical and diagnostic methods can have substantial value for clinical use.

In general, one aspect of this document features a method for assessing a soft tissue tumor present within a mammal. The method comprises, or consists essentially of, (a) determining whether or not a sample of the tumor comprises an amplified CPM nucleic acid sequence; and (b) diagnosing the mammal as having a malignant soft tissue tumor if the sample comprises the amplified CPM nucleic acid sequence and diagnosing the mammal as not having a malignant soft tissue tumor if the sample does not comprise the amplified CPM nucleic acid sequence. The mammal can be a human. The mammal can be diagnosed as having a well-differentiated liposarcoma/atypical lipomatous tumor if the sample comprises the amplified CPM nucleic acid sequence. The mammal can be diagnosed as not having a well-differentiated liposarcoma/atypical lipomatous tumor if the sample does not comprise the amplified CPM nucleic acid sequence. The determining step can comprise performing in situ hybridization. The in situ hybridization can be fluorescent in situ hybridization. The in situ hybridization can comprise contacting the sample with a nucleic acid probe set comprising at least three BAC clones selected from the group consisting of RP11-717F7, RP11-426B12, RP11-630N19, RP11-1104N20, RP11-103608, and RP11-927F2; at least three BAC clones selected from the group consisting of RP11-61F20, RP11-816C9, RP11-185H13, and RP11-450G15; at least two BAC clones selected from the group consisting of RP11-571M6, RP11-970A5, and RP11-258J5; or at least three BAC clones selected from the group consisting of RP11-258J5, RP11-143123, RP11-571M6, and RP11-455C23.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1 is a genetic probe map depicting the positions of probes for detecting MDM2 and CPM on chromosome 12.

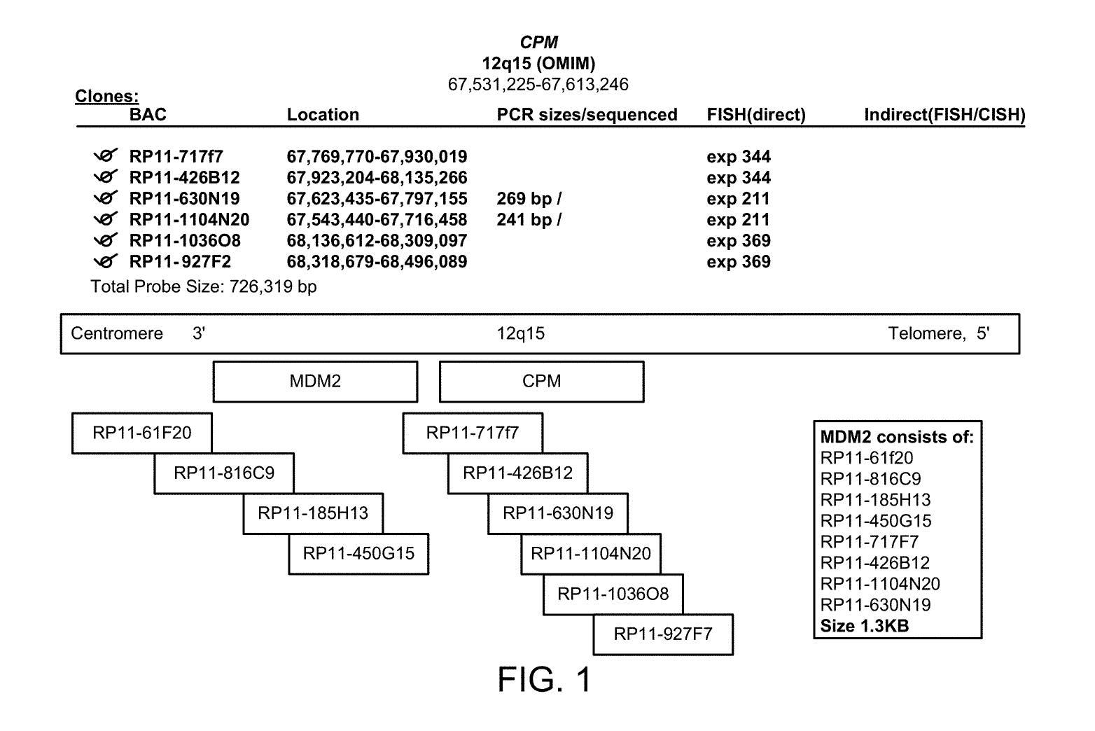

FIGS. 2A and 2B are photographs of ordinary lipoma and pleomorphic lipoma, respectively, probed with CPM. The presence of many CEP12/CPM signals, ratio=1, is observed in pleomorphic lipoma. FIG. 2C is a photograph of WDL/ALT showing MDM2 amplification. Similar to MDM2, CPM amplification is not seen in FIGS. 2A and 2B.

FIGS. 3A and 3B are photographs of a well-differentiated liposarcoma/atypical lipomatous tumor (WDL/ALT) exhibiting MDM2 and CPM amplification, respectively.

### DETAILED DESCRIPTION

This document provides methods and materials involved in assessing gene amplifications. For example, this document provides methods and materials for determining whether or not a sample from a mammal (e.g., a human) contains a ring or giant chromosome gene amplification. This document also provides methods and materials for determining whether or not a sample from a mammal (e.g., a human) contains amplification at a CPM, MDM2, CDK4, or TSPAN31 locus, or any combination thereof. Identifying such gene amplifications can be used to classify a mammal as having a lipomatous neoplasm and to distinguish between types of lipomatous neoplasms.

The term “nucleic acid” as used herein can be RNA or DNA, including cDNA, genomic DNA, and synthetic (e.g., chemically synthesized) DNA. The nucleic acid can be double-stranded or single-stranded. Where single-stranded, the nucleic acid can be the sense strand or the antisense strand. In addition, nucleic acid can be circular or linear.

The term “isolated nucleic acid” as used herein includes any non-naturally-occurring nucleic acid sequence since such non-naturally-occurring sequences are not found in nature and do not have immediately contiguous sequences in a naturally-occurring genome. An isolated nucleic acid can be, for example, a DNA molecule, provided one of the nucleic acid sequences normally found immediately flanking that DNA molecule in a naturally-occurring genome is removed or absent.

This document provides a collection of nucleic acid molecules (e.g., probes) having the ability to detect the presence of supernumerary ring chromosomes and/or giant rod chromosomes formed from amplified regions of chromosome 12 and others. Collections of such nucleic acid molecules can contain at least one (e.g., 2, 3, 4, 5, 10, or more) nucleic acid molecule having the ability to hybridize to amplified nucleotide sequence from chromosome bands 12q13→q15. For example, a collection of isolated nucleic acid molecules provided herein can contain at least one isolated nucleic acid molecule having the ability to hybridize to a CPM nucleotide sequence and/or at least one isolated nucleic acid molecule having the ability to hybridize to a MDM2 nucleotide sequence. The sequences of nucleic acid molecules (e.g., probes) can be derived from human genomic DNA. Examples of isolated nucleic acid molecules having the ability to hybridize to a CPM nucleotide sequence include, without limitation, RP11-717F7, RP11-426B12, RP11-630N19, RP11-1104N20, RP11-1306808, and RP11-927F7. Examples of isolated nucleic acid molecules having the ability to hybridize to a MDM2 nucleotide sequence include, without limitation, RP11-61F20, RP11-816C9, RP11-185H13, and RP11-450G15. Examples of isolated nucleic acid molecules having the ability to hybridize to a CDK4 nucleotide sequence include without limitation, RP11-571M6, RP11-970A5, and RP11-258J5. Examples of isolated nucleic acid molecules having the ability to hybridize to a TSPAN31 nucleotide sequence include, without limitation, RP11-258J5, RP11-143123, RP11-571M6, and RP11-455C23. Collections of isolated nucleic acid molecules having the ability to detect gene amplifications can include a vector such as a bacterial artificial chromosome (BAC) or a fosmid. For example, a collection of nucleic acid molecules provided herein can be a collection of BACs containing nucleotide sequences capable of hybridizing to, for example, a CPM nucleic acid sequence or a MDM2 nucleic acid sequence. Isolated nucleic acid molecules having the ability to detect gene amplifications can be any length. In some cases, isolated nucleic acid molecules provided herein (e.g., nucleic acid molecules having the ability to detect gene amplifications) can be more than 50 base pairs (bp) in length (e.g., more than 100 bp, 250 bp, 500 bp, 1 kb, 2 kb, 5 kb, 7 kb, 10 kb, 20 kb, 50 kb, 100 kb, 200 kb, or 300 kb). Isolated nucleic acid molecules provided herein can have sequences that overlap with another member of the collection. In some cases, each nucleic acid molecule of a collection can have a sequence that is distinct from the sequences of the other members of the collection. The isolated nucleic acid molecules of the collections provided herein can hybridize to CPM, MDM2, CDK4, or TSPAN31 nucleotide sequences present in either an intron or an exon. Introns and exons to which isolated nucleic acid molecules having the ability to detect gene amplifications can hybridize can be upstream or downstream of the transcription start site or the termination codon of a CPM, MDM2, CDK4, or TSPAN31 nucleotide sequence.

One or more of the isolated nucleic acid molecules provided herein can be labeled (e.g., fluorescently, biotin-labeled, antigen-labeled, or radioactively labeled) and used as probes (e.g., fluorescent in situ hybridization (FISH) probes). In some cases, the collections of isolated nucleic acid molecules provided herein can be labeled with a fluorophore (e.g., SpectrumGreen™ or SpectrumOrange™ (Vysis, Inc., IL)). SpectrumOrange™-labeled nucleic acid can be used to generate a signal that can be referred to as red (“R”). SpectrumGreen™-labeled nucleic acid can be used to generate a signal that can be referred to as green (“G”). SpectrumAqua™-labeled nucleic acid can be used to generate a signal that can be referred to as aqua (“AQ”). Proximal signals from SpectrumOrange™-labeled nucleic acid and SpectrumGreen™-labeled nucleic acid can combine to form a fusion (“F”) signal. Fusion signals can be distinguishable from other signals as adjacent red and green signals or fusion signals can appear as a combined red-green signal (e.g., yellow). It will be understood that the fluorophores used herein can be substituted with alternative sets of distinguishable fluorophores. For example, to detect co-amplification by two- or three-color FISH, the probe for each locus can have a detectable label of a different fluorochrome label. In such a case, fluorescence microscopy can be performed to excite and detect multiple fluorophores.

In situ hybridization using the nucleic acids provided herein can be performed using any appropriate technique, such as interphase, metaphase, or fiber FISH. For example, amplified sequences of the ring and giant rod chromosomes can be detected by fluorescent in situ hybridization (FISH). Generally, FISH is a method for detecting RNA or DNA sequences in cells, tissues, and tumors. For cytogenetic identification of lipomatous neoplasms, FISH can be used to visualize specific segments of DNA on metaphase chromosomes. For example, single-stranded nucleic acid probes can be contacted to a tissue sample such that nucleic acid hybrids or complexes form between complementary sequences. An exemplary FISH technique for the methods and materials provided herein can include (1) fixation of a specimen on a microscope slide; (2) hybridization of a labeled probe to homologous fragments of genomic DNA; and (3) enzymatic detection of the tagged target hybrids. In some cases, extra chromosomal material indicative of amplification of individual ring or giant rod chromosomes can be detected according to the methods described herein. In some case, co-amplification of more than one ring or giant rod chromosome gene can be detected. For example, amplification at the CPM, MDM2, CDK4, or TSPAN31 loci can be detected according to the methods described herein. In some cases, co-amplification at the CPM and MDM2 loci can be detected according to the methods described herein.

Any appropriate sample can be used for the detection techniques described herein. For example, such techniques can be performed on cells of fresh-fixed or paraffin-embedded tissue samples. Cells from any tissue source can be used, including biopsy tissues. Microscopy can then be used to detect the presence or absence of a gene amplification. The pattern and size of a signal can be used to estimate the location of a gene amplification.

The methods provided herein can be used to determine whether a mammal has a benign or a malignant lipomatous neoplasm. Methods for determining whether a mammal has a malignant lipomatous neoplasm can include identifying a mammal suspected of having a malignant lipomatous neoplasm and determining from a tissue sample from that mammal the presence or absence of cells having a gene amplification. The presence or absence of cells having a gene amplification can be determined by hybridizing nucleic acid from a tissue sample of the mammal with an in situ probe for CPM, MDM2, CDK4, or TSPAN31 and evaluating the presence or absence of co-amplification at such genetic loci. In some cases, a mammal suspected of having a lipomatous neoplasm can exhibit a known clinical symptom of a lipomatous neoplasm including, but not limited to, swelling, mass formation, soreness, and localized pain. A mammal exhibiting a known clinical symptom of a lipomatous tumor and that is found to have a gene amplification or co-amplification at a CPM, MDM2, CDK4, or TSPAN31 locus as compared to a mammal that does not have a lipomatous neoplasm, can be classified as having a lipomatous neoplasm. A mammal that exhibits no clinical symptoms of a lipomatous tumor but that is found to have a gene amplification or co-amplification at a CPM, MDM2, CDK4, or TSPAN31 locus as compared to a mammal that does not have a lipomatous neoplasm, can be classified as having a lipomatous neoplasm on the basis of such gene amplification.

As described herein, the presence or absence of co-amplification of CPM and MDM2 can permit identifying and classifying a malignant lipomatous neoplastic tissue in a sample. For example, detecting an amplified product of a particular size can indicate the presence and/or identity of tissue as having a well-differentiated liposarcoma/atypical lipomatous tumor (WDL/ALT), a lipomatous tumor, or no adipose neoplasm. MDM2 is amplified in greater than 99% of WDL/ALTs, and is amplified in up to 30% of other sarcomas. CPM is consistently co-amplified with MDM2 in WDL/ALT. In some cases, therefore, detecting co-amplification of CPM and MDM2 in a sample is indicative of a sample containing a lipomatous neoplasm identified as a well-differentiated liposarcoma/atypical lipomatous tumor. In some cases, the absence of co-amplification of CPM and MDM2 is indicative of the absence of WDL/ALT in a sample once it is recognized as of adipose tissue differentiation.

The detection methods described herein can be performed in combination with other methods of identifying adipose neoplasms. For example, detecting the presence or absence of co-amplification of CPM and MDM2 can be performed in combination with histologic evaluation to aid identifying and classifying a sample as having a particular adipose neoplasm.

In some cases, detection of amplification at CPM and MDM2 loci in a sample can enable clinicians or other professionals to classify a mammal as possessing a WDL/ALT. Information collected according to the methods provided herein can be used to assess the health state of a mammal (e.g., a human patient), such as presence or absence of a disorder (e.g., malignant lipomatous neoplasm) or to evaluate risk of developing such a disorder. In some cases, results of the ring and giant rod chromosome amplification detection methods and materials provided herein can be communicated by research technicians or other professionals who perform the detection assay to clinicians or other professionals who will classify the mammal as having a particular pathology. For example, a researcher or diagnostician can communicate information regarding the presence or absence of a WDL/ALT to a clinician or other medical professional. Any appropriate method can be used to communicate input information regarding the presence or absence of a lipomatous neoplasm to another person (e.g., a professional), and information can be communicated directly or indirectly. For example, a laboratory technician can input information regarding the presence or absence of a lipomatous neoplasm into a computer-based record. In some cases, information can be communicated by making a physical alteration to medical or research records. For example, a medical professional can make a permanent notation or flag a medical record for communicating a diagnosis to other health-care professionals reviewing the record. Any type of communication can be used (e.g., mail, e-mail, telephone, and face-to-face interactions). Information also can be communicated to a professional by making that information electronically available to the professional. For example, information can be placed on a computer database such that a health-care professional can access the information. In addition, information can be communicated to a hospital, clinic, or research facility serving as an agent for the professional.

In some cases, the methods described herein can include selecting a treatment regimen for a subject determined to have a malignant soft tissue tumor or a well-differentiated liposarcoma/atypical lipomatous tumor based upon the presence of amplification at CPM and MDM2 loci as described herein. For example, clinicians or other professionals can initiate or modify a treatment regimen after receiving information regarding detection of amplification at CPM and MDM2 loci as described herein. The determination of a treatment regimen can also be based upon the absence or presence of other risk factors associated with malignant soft tissue tumors such as local recurrences and metastases. The methods can also include administering a treatment regimen to a subject having a malignant soft tissue tumor or well-differentiated liposarcoma/atypical lipomatous tumor to thereby treat, prevent, or delay further progression of the disease. As used herein, the term “treat” or “treatment” is defined as the application or administration of a treatment regimen, e.g., a therapeutic agent or modality, to a subject, e.g., a patient. For example, standard treatment regimens for malignant lipomatous neoplasm can include surgical excision of the neoplasm and injection of compounds that trigger lipolysis (e.g., steroids, phosphatidylcholine) or target neoplastic cells (e.g., antibody therapy, radiation therapy, chemotherapy). In some cases, treating can include eliminating, preventing the regrowth, and inhibiting proliferation of lipomatous cells in a subject diagnosed as having a malignant soft tissue tumor. For example, methods of treating can include administering compositions or compounds that effectuate the elimination, prevention of regrowth, or inhibition of lipomatous cells. Such compositions can include alkylating agents, tyrosine kinase inhibitors, and MDM2 polypeptide. The invention will be further described in the following examples, which do not limit the scope of the invention described in the claims.

### EXAMPLES

### Example 1

### Probes for Detecting CPM MDM2, CDK4, and TSPAN31

Bacterial artificial chromosome (BAC) clones spanning CPM, MDM2 loci located at12q15 and CDK4 located at loci on 12q14.1 and TSPAN31 located at loci 12q14.1, were obtained from Children's Hospital Oakland Research Institute (Oakland, Calif.). BAC clones for MDM2 were: RP11-61F20, RP11-816C9, RP11-185H13, and RP11-450G15. BAC clones for CPMwere: RP11-717F7, RP11-426B12, RP11-630N19, RP11-1104N20, RP11-1036O8, and RP11-927F2. BAC clones for CDK4 were: RP11-571M6, RP110970A5, and RP11-258J5. BAC clones for TSPAN31 were: RP11-258J5, RP11-143I23, RP11-571M6, and RP11-455C23. Each of the probe sets (Table 1) was mixed with the Cep 12 (D12Z3) SpectrumGreen probe (12q11.1-q11) (Abbott Laboratory, North Chicago, Ill.). All of the identities of the BAC clones were individually confirmed by PCR and by hybridization on metaphase preparations from the peripheral blood of five normal individuals. Their performance on paraffin embedded tissues was verified on numerous normal tissue types, including skeletal muscle, adipose tissue, gastrointestinal mucosa, brain, and others. Normal structures that can be readily identified under the 4,6-diamidino-2-phenylindole (DAPI) staining on thin sections, such as blood vessels and epidermis, were used as internal controls for the cases analyzed.

DNA isolation was performed using the Qiagen Plasmid Maxi Kit (Qiagen, Valencia, Calif.). DNA was labeled using a nick translation kit (Abbott Laboratory, North Chicago, Ill.). Interphase molecular cytogenetic studies were performed using 4-μm paraffin-embedded thin sections that were deparaffinized twice in xylene (15 minutes per treatment), dehydrated twice in 100% ethanol (5 minutes per treatment), and treated with 10 mmol/L citric acid (10 minutes, in a humidified microwave). Tissue sections were incubated in warm (37° C.) sodium chloride-sodium citrate buffer (2×SSC) for 5 minutes. Protein was digested with Digest-All 3 (Invitrogen Corporation, Carlsbad, Calif.). After a brief wash in PBS (1×PBS), slides were sequentially dehydrated in ethanol (70, 85, and 100%) and air-dried at room temperature. Tissue sections were denatured at 85° C. for 5 minutes, and BAC probe hybridization was performed overnight in a humidified chamber at 37° C. Tissue sections were washed in 0.1% NP40 (NP40) in 2×SSC at 76° C. for 2 minutes and then washed in the same solution at room temperature for 2 minutes. Slides were mounted in Vectashield mounting medium (Vector Laboratories, Burlingame, Calif.) with 1.5 μg/mL of DAPI medium (Vector Laboratories). Tumor samples were considered positive if more than 10% of the 200 cells analyzed exhibited amplification from the FISH probes. Tumors were evaluated and scored by two independent technologists.

TABLE 1

| | | GenBank Accession and | GenBank Accession | | | |
|---|---|---|---|---|---|---|
| | | GI Numbers for | and GI Numbers for | First | Last | |
| | Probe Set | Beginning End of BAC | Ending End of BAC | nucleotide | nucleotide | Probe |
| Probe Set | (BAC clones) | Clone | Clone | position | position | length |
| CPM | | | | | | 726,319 |
| | RP11-717F7 | AQ512835; 4745126 | AQ431497; 4541832 | 67,769,770 | 67,930,019 | |
| | RP11-426B12 | AQ553131; 4912308 | AQ553135; 4912312 | 67,926,208 | 68,135,240 | |
| | RP11-630N19 | AQ435945; 4547284 | AQ441428; 4552767 | 67,623,435 | 67,797,155 | |
| | RP11-1104N20 | AQ811882; 5772860 | AQ719218; 5478887 | 67,543,440 | 67,716,458 | |
| | RP11-1036O8 | AQ673038; 5205784 | AQ672960; 5205706 | 68,136,612 | 68,309,097 | |
| | RP11-927F2 | AQ666000; 5173768 | AQ770956; 5648995 | 68,318,679 | 68,496,089 | |
| MDM2 | | | | | | 1,152,153 |
| | RP11-61F20 | AQ196363; 3607975 | AQ196362; 3607974 | 66813948 | 66975069 | |
| | RP11-816C9 | AQ597140; 5028352 | AQ516176; 4748434 | 66965960 | 67185000 | |
| | RP11-185H13 | AQ419678; 4477402 | AQ419692; 4477416 | 67160267 | 67363897 | |
| | RP11-450G15 | AQ632414; 5095049 | AQ586521; 5013567 | 67366758 | 67543065 | |
| CDK4 | | | | | | 612,651 |
| | RP11-571M6 | AC025165.27; 12000427 | | 56286137 | 56497675 | |
| | RP11-970A5 | AQ801330; 5718662 | AQ815678; 5778071 | 56422556 | 56639338 | |
| | RP11-258J5 | AQ479738; 4661857 | AQ479736; 4661855 | 56,026,687 | 56,215258 | |
| TSPAN31 | | | | | | 794,010 |
| | RP11-258J5 | AQ479738; 4661857 | AQ479736; 4661855 | 56,026,687 | 56,215,258 | |
| | RP11-143I23 | AQ388827; 4359850 | AQ388812; 4359835 | 56,395,555 | 56,569,128 | |
| | RP11-571M6 | AC025165.27; 12000427 | | 56,286,137 | 56,497,675 | |
| | RP11-455C23 | AQ587057; 5013793 | AQ587059; 5013795 | 56,587,628 | 56,721,821 | |

### Example 2

### Detecting Amplification at the CPM Locus

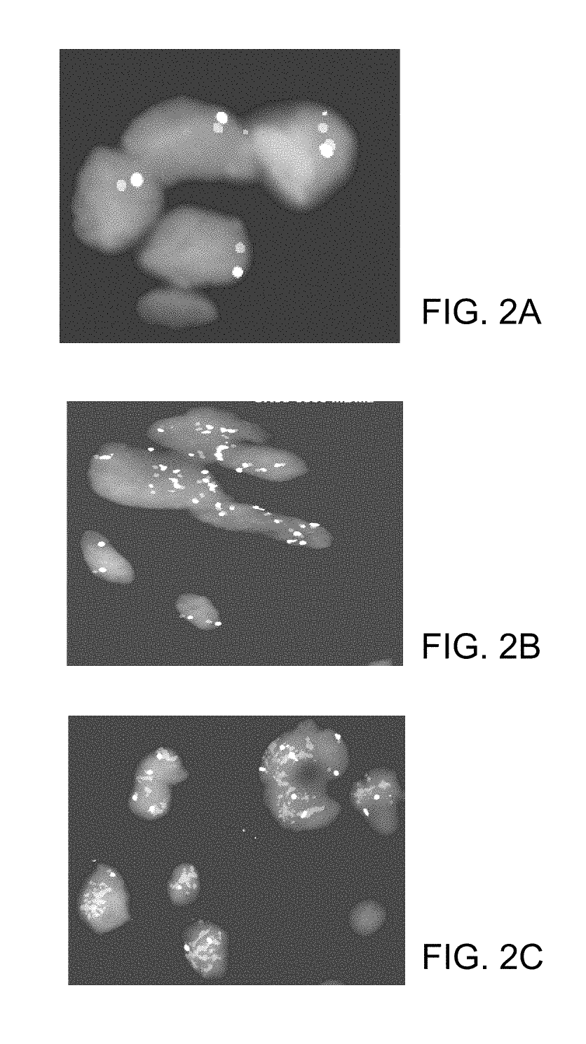

For cytogenetic identification of adipose neoplasms in a tissue sample, amplification at the CPM locus was detected in tissue samples. FISH was performed on fresh formalin-fixed and paraffin-embedded tissue samples as described elsewhere (Cataldo et al., Am. J. Surg. Pathol. 23(11):1386-92 (1999)). Formalin-fixed, paraffin-embedded tissues were mounted on glass slides. Slides were prepared, with some slides stained with hematoxylin and eosin (H&E) and the remaining slides left as unstained slides. The selection of tissue and the identification of target areas on an H&E-stained slide were performed. Using the H&E slide as a reference, target areas were etched with a diamond-tipped etcher on the back of the unstained slide to be assayed. Abnormalities involving the CPM locus at 12q13→15 were detected using FISH genetic mapping probe presented in Table 1, along with a reference probe, CEP 12 (Abbott Molecular). Probe sequences were derived from bacterial artificial chromosomes (BACs) spanning the CPM locus region and labeled with fluorescent label Spectrum Orange™ (“R”). The reference probe, CEP 12 (D12Z3), was labeled with fluorescent label Spectrum Green™ (“G”). The probe set was applied to the appropriate target areas, denatured, and hybridized overnight. Interphase nuclei were analyzed by fluorescence. Normal interphase nuclei showed 2R+2G signals. Normal patterns also included 1R+1G, 1R+2G, and 2R+1G (FIG. 2A). Abnormal nuclei (i.e., those nuclei bearing amplification at the CPM locus) exhibit 3 or more additional G signals with amplification of the R signal (FIG. 2C). Cells having multiple copies of RG at a ratio of 1:1 were considered abnormal, but were not treated as a “positive” result for CPM amplification (FIG. 2B). If a result was comparable between two or more scorers, the result was scored as positive or negative for amplification of the CPM locus 12q13→15.

### Example 3

### Co-Amplification Validation Assay

Seventeen WDL/ALT, 22 ordinary lipomas, and 16 other tumors, including 6 myxoid liposarcomas, 4 pleomorphic lipomas, 4 pleomorphic liposarcomas, and one each of lipomatous variant of angiomyofibroblastoma and a high grade undifferentiated pleomorphic liposarcoma were evaluated by MDM2 and CPM amplification using fluorescent in situ hybridization (FISH) on 4 μm paraffin-embedded tissue sections. All experiments were performed by co-hybridizing MDM2 or CPM (custom designed probes) with a commercially available centromere 12 specific probe (CEP12 (D12Z3, Vysis®)). Signal pattern evaluation was performed on 200 cells/tumor by two technologists without prior knowledge of the histological diagnosis.

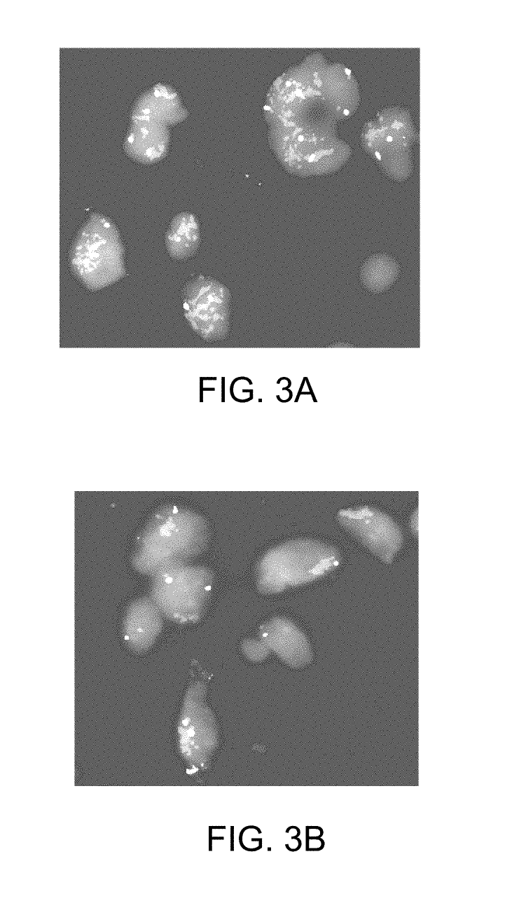

All WDL/ALT were found to have amplification of both MDM2 and CPM (100%) (usually>20 copies/cell). Lipomas and the lipomatous variant of angiomyofibroblastoma demonstrated normal signal patterns with only two copies of MDM2 and CPM. All pleomorphic tumors (lipoma and liposarcoma) exhibited FISH signal patterns consistent with aneuploidy without amplification of either CPM or MDM2. Two of 6 myxoid liposarcomas exhibited patterns consistent with monosomy 12 or loss of the CPM/MDM2 loci, while the remaining four exhibited normal FISH signal patterns. Well-differentiated liposarcoma/atypical lipomatous tumor (WDL/ALT) exhibiting MDM2 and CPM amplification are shown in FIGS. 3A and 3B, respectively.

CPM was co-amplified with MDM2 amplification in 100% of WDL/ALT but in none of the other tumors evaluated, including 22 ordinary lipomas. These data suggest that FISH for CPM amplification can be used as a diagnostic tool for the diagnosis of lipomatous neoplasms.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. (canceled)

2. A fluorescent in situ hybridization (FISH) method comprising:
(a) obtaining a sample of cells from a mammal, wherein said sample comprises an adipose neoplasm,
(b) co-hybridizing an MDM2 (mouse double minute 2 homolog) FISH probe and a centromere 12 FISH probe to a first portion of said sample of cells,
(c) detecting signals from said MDM2 FISH probe and said centromere 12 FISH probe following said co-hybridizing of step (b),
(d) determining, from said signals of step (c), if MDM2 nucleic acid of said mammal is amplified,
(e) co-hybridizing a CPM (carboxypeptidase M) FISH probe and a centromere 12 FISH probe to a second portion of said sample of cells,
(f) detecting signals from said CPM FISH probe and said centromere 12 FISH probe following said co-hybridizing of step (e), and
(g) determining, from said signals of step (f), if CPM nucleic acid of said mammal is amplified.

3. The method of claim 2, wherein said mammal is a human.

4. The method of claim 2, wherein said cells are well-differentiated liposarcoma cells or atypical lipomatous tumor cells.

5. The method of claim 2, wherein said determining step (d) comprises determining that said MDM2 nucleic acid of said mammal is amplified.

6. The method of claim 2, wherein said determining step (g) comprises determining that said CPM nucleic acid of said mammal is amplified.
